(19) United States
(12) Patent Application Publication
(10) Pub. No.: US 2004/0214247 A1
(43) Pub. Date: Oct. 28, 2004

(54) Novel fibronectin epitopes and proteinaceous molecules capable of binding said epitopes

(76) Inventors: Cornelis Adriaan De Kruif, El De Bilt (NL); Ton Logtenberg, Werkhoven (NL); Marinus Theodorus Thomas Pennings, Utrecht (NL); Henriette Christine De Boer, Zeist (NL)

Correspondence Address: TRASK BRITT, P.O. BOX 2550, SALT LAKE CITY, UT, 84110, US

(21) Appl. No.: 10/466,466

(22) PCT Filed: Jan. 18, 2002
(86) PCT No.: PCT/NL02/00042

Related U.S. Application Data
(60) Provisional application No. 60/263,028, filed on Jan. 19, 2001.

(30) Foreign Application Priority Data
Jan. 19, 2001 (EP) 01200212.7

(51) Int. Cl. G01N 33/574; A61K 39/395; C07K 16/40

(52) U.S. Cl. 435/7.23; 424/146.1; 530/388.26

(57) ABSTRACT

Means and method for at least partial inhibition of tumor growth are provided. Methods makes use of binding molecules capable of specifically binding to an epitope present on a subset of fibronectin proteins. By providing an individual with a binding molecule of the invention it is possible to interfere with sites of angiogenesis or sites that seen active angiogenesis in the recent past. Through this interference blood flow is at least in part inhibited. Through this inhibition it is possible to at least in part inhibit processes dependent on active angiogenesis in said individual, such as tumor growth.

The invention relates to the field of medicine, more specifically to the treatment of cancer. The invention relates to antibodies, and particularly to antibodies that interact with human fibronectin.

The extracellular matrix controls cell survival, cell morphology and tissue organization by supporting cell adhesion. Remodeling of the extracellular matrix and cell migration are key processes in the development of properly organized blood vessels, tissues and organs, and have been implicated in pathological processes (Werb, 1997; Liotta et al., 1991). Many extra-cellular matrix proteins, such as fibronectin and members of the collagen family, and proteases, such as plasmin and metalloproteinases, are involved in the remodeling process. These proteins are implicated in modulation of tumor phenotype by affecting crucial processes such as cell migration, survival and/or angiogenesis, which is the formation of new blood vessels.

Fibronectin is a high molecular weight glycoprotein expressed at the cell surface of many types of differentiated cells and is involved in the attachment of cells to the surrounding extracellular matrix. Fibronectin has affinity to the other main components of extracellular matrix, collagen and glycosaminoglycans. It also interacts with cell surfaces as shown by the finding that fibronectin-collagen complexes, or fibronectin alone when insolubilized on a surface such as plastic, enhances the attachment of various types of cells to such surfaces. Conformational flexibility has the potential to influence the biological properties of fibronectin. Soluble plasma fibronectin binds poorly to many cell types, but after deposition onto a suitable substrate, its cell binding avidity is enhanced. The substrates may be collagen in the extracellular matrix or fibrin in periferal blood. In the extracellular matrix, fibronectin provides signals that control cell shape, migration, proliferation, differentiation, morphogenesis and survival. This makes fibronectin a paradigm adhesive protein, non-reactive in its soluble state, but highly adhesive when insoluble. In the extracellular matrix, adhesive proteins display biologically active cryptic sites that are revealed after structural or conformational alterations of these molecules and are called matricryptic sites (Davis et al, 2000). This is in agreement with studies by Pickford et al (2001), who showed that collagen binding may induce a conformational change in fibronectin that disengages the intramolecular interaction of domains I-1/I-2/I-3/I-4/I-5 with III-3. As a result cryptic sites in this gelatin binding domain of fibronectin may be exposed.

Fibronectin is a dimer and consists of large disulfide-linked subunits composed of multiple structural domains. More than half of the molecule consists of so-called fibronectin type III repeats. Two splice-variants of fibronectin are known. A so-called ED-A splice variant is expressed in some normal tissues and can also be found in the serum. ED-A expression is upregulated in tumor tissue and embryonic tissue. A so-called ED-B splice variant is only expressed in embryonic tissue and tumor tissue. It is not detectable in healthy adult tissue (Reza Farnoud et al. 1995; Pujuguet et al. 1996).

To date many different diseases are being treated with antibodies. Nevertheless, many disorders are not being treated since no specific epitopes are found, that are expressed on cells or surfaces that need to be removed by the immune system through the interaction with antibodies or other proteinaceous molecules capable of binding such epitopes. Novel epitopes on and binding molecules to fibronectin, which is implicated in tumor phenotype and angiogenesis, are interesting candidates to diagnose and/or treat diseases.

### SUMMARY OF THE INVENTION

The current invention in one aspect provides a novel epitope on fibronectin, and in another aspect provides proteinaceous molecules preferentially binding said epitope. The epitope is different from the ED-A, ED-B, and oncofetal epitopes described earlier. The expression pattern throughout the body is not reminiscent of either of these described epitopes. The observed expression pattern of the epitope of the invention in a human body is unique and offers new opportunities for the diagnosis and/or the treatment of diseases. Expression of an epitope of the invention is associated with at least certain tumor cells, certain endothelial cells in the vicinity of tumor cells and extra-cellular matrix in the vicinity of tumor cells. It is postulated that the epitope of the invention is correlated with areas of active angiogenesis or areas where angiogenesis has occurred in the recent past. The epitope of the invention is preferentially recognized by a proteinaceous molecule according to the invention when fibronectin is bound to gelatin (either plasma containing fibronectin or fibronectin purified from plasma by gelatin-Sepharose affinity chromatography). A weak binding was observed when fibronectin was coated directly to plastic. Although the fibrillar form of collagen is known to bind fibronectin, it did not induce expression of the epitope of the invention. This indicates that a degraded/denatured form of collagen (like gelatin) is required to transform fibronectin into a different conformer, leading to expression of the conformational epitope of the invention. The proteinaceous molecule according to the invention also binds to a human dermal microvascular endothelial cell line (HDMEC), either in monolayer or brought in suspension after treatment with EDTA. Apparently, this endothelial cell-bound form of fibronectin also displayed the conformational change-that leads to exposure of the epitope of the invention.

### DETAILED DESCRIPTION

In one aspect the invention provides a proteinaceous molecule capable of binding to fibronectin upon the interaction between fibronectin and one or more of gelatin, fragmented collagen and denatured collagen. Denatured and/or degraded/fragmented collagen can be generated by proteolysis, due to acid, high temperatures, proteases, combinations thereof, and the like. Gelatin-comprises denatured collagen-rich material. The binding of said proteinaceous molecule to fibronectin can take place when fibronectin interacts with gelatin and/or fragmented collagen, and/or denatured collagen, and not or to a much lesser extent when fibronectin does not interact with one of said substances. Hence said proteinaceous molecule preferentially binds fibronectin under one of said conditions, or binding to fibronectin is induced or enhanced under one of said conditions. Interaction of fibronectin to one of said substances includes but is not necessarily limited to direct binding of fibronectin to said substances. The proteinaceous molecule according to the invention does bind fibronectin directly coated to plastic, but this binding is strongly enhanced when fibronectin interacts with gelatin. Therefore, it is an aspect of the present invention to provide a proteinaceous molecule capable of binding to fibronectin, wherein said binding is enhanced by the interaction between fibronectin and one or more of gelatin, fragmented collagen and denatured collagen. In one aspect, said binding is enhanced by a factor of at least two, preferably at least twenty. In another embodiment said proteinaceous molecule binds to an epitope that is induced by the interaction between fibronectin and one or more of gelatin, fragmented collagen and denatured collagen. Said epitope is induced or exposed upon interaction of fibronectin with one of said substances, and is exposed to a much lesser extent or masked when fibronectin does not interact with one of said substances. Induction of epitopes takes place when cryptic epitopes are revealed e.g. after structural conformation changes. The epitope of the invention is not expressed in any of the tested normal human tissues. It is however, expressed on some types of tumor cells, some kinds of endothelial cells in the vicinity of tumor cells and extra-cellular matrix in the vicinity of tumor cells. Therefore, in one aspect, the epitope is tumor-specific. Tumor-specific is meant to include preferential expression in tumor tissues compared to normal tissues. The epitope of the invention is also expressed in a human microvascular endothelial cell line and not in a macrovascular endothelial cell line. Therefore, in another aspect, induction of the epitope coincides with angiogenesis. In the extracellular matrix, adhesive proteins display biologically active cryptic sites that are revealed after structural or conformational alterations of these molecules and are called matricryptic sites (Davis et al, 2000), and epitopes that are thus revealed are herein referred to as matricryptic epitopes. It is an aspect of the invention to provide a proteinaceous molecule capable of binding to a matricryptic epitope in fibronectin. In another aspect, said matricryptic epitope is induced upon interaction of fibronectin with one or more of gelatin, fragmented collagen or denatured collagen. In one embodiment, said matricryptic epitope is tumor-specific. Mapping of the epitope that binds the proteinaceous molecule of the invention revealed that the epitope is at least in part in a 40 kDa fragment of fibronectin, that is implicated in gelatin/collagen-binding. Therefore, in one aspect, an epitope of the invention is at least in part in the gelatin-binding domain of fibronectin.

In one embodiment the proteinaceous molecule of the invention is capable of distinguishing a subset of fibronectin containing matrices and/or fibronectin containing cells. Binding of a proteinaceous molecule capable of specifically binding to an epitope present in a subset of fibronectin proteins can be used in a diagnostic setting. For instance, to determine whether tumor tissue is present in a sample taken from a patient, or to detect or image areas of active angiogenesis. The binding of said proteinaceous molecule to its target can also be used in therapeutic settings. In a preferred embodiment the invention provides a method for at least in part preventing growth of tumor cells comprising providing said tumor cells and/or surrounding matrix tissue with a proteinaceous molecule capable of specifically binding to an epitope present in a subset of fibronectin proteins. Tumor growth is at least in part prevented through direct interaction of said proteinaceous molecule with said subset of fibronectin proteins. Fibronectin not belonging to the subset is not bound by said proteinaceous molecule and, therefore, not subject to the effects of the proteinaceous molecule. Without wishing to be bound by theory it is believed that binding of said proteinaceous molecule interferes with one or more signaling properties of fibronectin or fibronectin-associated molecules. Fibronectin, especially in provisional extracellular matrix in areas where blood vessel formation occurs, facilitates such blood vessel formation. This facilitation can at least in part be interfered with, by allowing a proteinaceous molecule of the invention to bind to said fibronectin. Preferably, the epitope for binding of the proteinaceous molecule is at least in part specific for the tissue where blood vessel formation is to be at least in part inhibited. Preferably, said epitope comprises an epitope of the invention. The distribution of an epitope of the invention is particularly favorable for use in therapeutic settings not in the least because it has not been detected in any of the normal tissues tested yet. By providing an individual with a binding molecule of the invention it is possible to interfere with sites of angiogenesis or sites that were active in angiogenesis in the recent past. Through this interference blood flow is at least in part inhibited. Through this inhibition it is possible to at least in part inhibit processes dependent on active angiogenesis in said individual, such as tumor growth. It is therefore an aspect of the invention to use a proteinaceous molecule according to the invention for detection, imaging or inhibition of angiogenesis. It is another aspect to use a proteinaceous molecule according to the invention for the detection of the onset of angiogenesis and/or a physiological condition associated with angiogenesis in a human subject. The absence of an epitope of the invention in normal tissue allows systemic administration routes for the proteinaceous molecule of the invention. This is advantageous because it allows the use of the blood stream for dissemination of the proteinaceous molecule of the invention, with all the associated advantages (suitable penetration and distribution). A limited availability of the targeted epitope outside the area targeted with the therapy is usually tolerated. Blood vessel formation can be inhibited in a normal individual, for at least a limited period, without invoking serious side effects. Individuals in which angiogenesis should not be interfered with, such as pregnant women or women that want to conceive, might observe miscellaneous detrimental effects. If need be, patients in which side effects are anticipated can be excluded from receiving a fibronectin binding proteinaceous molecule of the invention.

In some embodiments of the invention the binding of a proteinaceous molecule of the invention can recruit immune system components to bound fibronectin. This recruitment facilitates removal of the bound extracellular matrix and/or the bound cell. Alternatively, said proteinaceous molecule may comprise or be provided with a tag. Preferably said tag is capable of interfering with cellular processes. With said preferred tag said proteinaceous molecule is capable of at least in part improving the anti-tumor capabilities of a proteinaceous molecule of the invention. Preferably, said tag comprises an angiogenesis inhibitor, a toxin, a cytostatic drug and/or a radioactive compound.

In one aspect of the invention, said proteinaceous molecule is capable of binding to a neoplastic cell. Such binding can be used to mark said cell in a diagnostic assay or in a therapeutic setting. For instance, binding of a proteinacous molecule of the invention may trigger the death of the bound cell. In one embodiment, said neoplastic cell is derived from a breast cell, prostate cell, colon cell, lung cell, skin cell, pancreas cell, bladder cell, head cell or a neck cell. In another aspect said proteinaceous molecule is capable of binding fibronectin in a microvessel. In one aspect said fibronectin is part of an extracellular matrix. In one embodiment said extracellular matrix is in the vicinity of a tumor cell. In another embodiment said proteinaceous molecule is capable of binding to a microvascular endothelial cell. Preferably, said microvascular cell lies in the vicinity of a tumor cell. Binding of a proteinaceous molecule of the invention to a microvascular cell can be used to at least in part prevent outgrowth of a new blood vessel thereby limiting the blood supply to tumor cells thereby at least in part limiting the growth of said tumor cells. An epitope of the invention is present in different types of animals. However, considering the clinical and diagnostic uses said proteinaceous molecule is preferably capable of binding to a subset of human fibronectin proteins.

Many different types of binding molecules are known in the art. For proteinaceous targets such as fibronectin, binding molecules are typically proteinaceous molecules. They at least comprise some amino-acids linked through a peptide linkage. Typically such proteinaceous binding molecules comprise at least 7 amino-acids that are linked through peptide linkage. A proteinaceous molecule of the invention can be found for instance using phage display of peptides. In a preferred embodiment of the invention said proteinaceous molecule is an antibody or a functional part or derivative thereof. A functional part of an antibody comprises at least one antigen binding part of said antibody. Non-limiting parts are F(ab) and F(ab)₂ fragments. A derivative or a part of an antibody comprises essentially the same antigen binding properties of a proteinaceous molecule of the invention. A derivative may comprise one or more amino-acid substitutions, insertions or deletions relative to an antibody or part thereof, of the invention. In another preferred embodiment said proteinaceous molecule is a single chain Fv fragment. A proteinaceous molecule according to the invention can be obtained by expression, using methods that are well known in the art, from a plasmid designated Fibmab-His/myc, deposited at the European Collection of Cell Cultures (ECACC) on 15 Jan. 2002, under accession number 02011518. Preferably, said antibody or functional part, or derivative thereof is human or humanized. Preferably, a proteinaceous molecule of the invention further comprises a tag. Preferably, said tag comprises a toxin and/or a radioactive substance. In a preferred embodiment said proteinaceous molecule comprises a heavy chain comprising a CDR3 region, said CDR3 region comprising the sequence EDTAVYYCAR NPFQSS FDYWGQ or a derivative and/or functional analogue thereof. In another preferred embodiment said proteinaceous molecule comprises a light chain comprising a CDR3.region, said CDR3 region comprising the sequence EDFATYYC SQFSTMPGGFGQGTK VEIK or a derivative and/or functional analogue thereof. A derivative and/or functional analogue of a heavy chain or a light chain mentioned above is a molecule comprising the same heavy or light chain activities in kind not necessarily in amount. A derivative or functional analogue may comprise one or more amino-acid substitutions, insertions or deletions relative to the sequence of the invention, and can easily be made and tested by the skilled person based on the sequence of the invention. Said heavy or light chain activities preferably include antigen-binding activities of the one or the combination of the chains.

In one aspect the invention provides a method for the treatment of an individual suffering from or at risk of suffering from a disease, comprising administering to said individual a therapeutically effective amount of a proteinaceous molecule of the invention. In one aspect, said disease is a neoplastic disease. In a similar aspect the invention provides the use of a proteinaceous molecule of the invention for the preparation of a medicament. In yet another embodiment the invention provides a method for typing a cell comprising determining whether said cell is capable of specifically binding a proteinaceous molecule of the invention. Similarly the invention also provides the use of an epitope expressed on a subset of fibronectin expressing cells as a marker for neoplastic cells, wherein said epitope is a tumor-specific matricryptic epitope. In another aspect the invention provides a cell recognized by a proteinaceous molecule according to the invention, wherein said cell exposes at least one matricryptic epitope on fibronectin.

With the current technology it is possible to obtain nucleic acid encoding a proteinaceous binding molecule. Particularly for peptides and antibody like binding molecules it is possible to obtain encoding nucleic acid. This can be done for instance through amplifying the selected phages in phage display approaches or the amplification of antibody encoding RNA in hybridoma approaches. The invention therefor further provides a nucleic acid encoding a proteinaceous molecule of the invention, or a part thereof involved in fibronectin binding of said molecule. For production of proteinaceous molecule of the invention encoding nucleic acid can be cloned into one or more suitable expression vectors and transferred to cells. The invention therefor also provides a cell comprising a nucleic acid of the invention. Preferably, said cell is a primate, rodent, bird or plant cell. Such cells are particularly suited for the production of binding molecules. Preferably, said cell is a human cell. In this way the binding molecule is produced by a human cell and should therefore comprise post-translational modifications that are compatible with use in humans. It has been observed that such binding molecules have improved pharmaco-dynamic properties in humans. In another preferred embodiment said cell further comprises nucleic acid encoding an early protein of adenovirus or a functional part, derivative and/or analogue thereof, wherein said early protein of adenovirus is E1 or E2A. Such cells are typically very well suited for obtaining high yields of protein, and typically show improved culture characteristics such as suspension growth and serum free adaptation.

In the field binding molecules are also produced using a plant or a non-human animal. The invention therefor also provides such production platforms. In a preferred embodiment said plant or non-human animal is transgenic for a nucleic acid of the invention.

In yet another aspect the invention provides a gene delivery vehicle comprising a proteinaceous molecule of the invention. Such gene delivery vehicles are targeted toward said subset of fibronectin proteins. Such a gene delivery vehicle can be used in a method for the treatment of an individual suffering from or at risk of suffering from a disease, comprising administering to said individual a therapeutically acceptable amount of a gene delivery vehicle of the invention.

Preferably said disease is a neoplastic disease. A gene delivery vehicle of the invention can favorably be used for the preparation of a medicament.

Provides is further a kit comprising a proteinaceous molecule and/or a gene delivery vehicle of the invention.

### EXAMPLES

### Example 1

### Phage selection on Human Microvascular Endothelial Cells (HDMEC-1)

T75 flasks, T175 flasks and 6 well plates were coated by overlaying the bottom overnight with a 1% gelatine/PBS solution. The flasks were then washed three times with PBS and stored with 10 ml PBS at 4° C. Endothelial cells were obtained from the Center for Disease Control and prevention (CDC, Atlanta, USA. Ades et al. 1992). The cells were grown on the gelatin coated T75 flasks in Dulbecco's Modified Eagle Medium (DMEM) with sodium pyruvate, 100 mM glucose and pyridoxine (Gibco) supplemented with 20% Human Pooled Serum (HPS) made from healthy donors, penicilline and streptomycin. The cells were further cultured in an incubator at 37° C., 5% CO₂.

The Endothelial cells were grown to 80-90% confluency in DMEM+20% HPS+pen/strep in a gelatine coated T75 flask and subsequently treated with trypsin in order to make a single cell suspension, washed with PBS and seeded in a 6 well plate at 20-30% confluency. Normally cells reached 65-70% confluency after 2 days of culturing and subsequently used for the phage selection procedure.

The scFv antibody phage display library used for selection procedures, is a semi-synthetic scFv library with a synthetic CDR3 region in the variable heavy chain derived from a semi-synthetic scFv library described by De Kruif et al. (1995). 500 μl of the scFv antibody phage library was incubated with 1.5 ml PBS containing 4% non-fat milk powder (Protifar), 1% gelatine, 20% HPS and 5×10⁸ Periferal Blood Cells (PBL's) for 30 minutes at 4° C. The Endothelial cells were then washed 6 times with 3 ml PBS at 4° C. and incubated with the previously described library mix for 4 hrs at 4° C. while shaking gently on a rocking platform. After this incubation and phage binding period, cells were washed extensively (20 times) with PBS+0.01% Tween20 and 20 times with PBS at 4° C. The Endothelial cells were subsequently harvested using a cell scraper and spun down at 1500 rpm. The cells and the phages bound to them were then incubated with 10 ml of a fresh XL1-blue bacteria culture (Stratagene) at OD₆₀₀ 0.5 and left at 37° C. for 30 min for infection of the bacteria by the phages. Bacteria were harvested by spinning at 3600 rpm and plated on TYE dishes (10 g/l Bacto-tryptone, 5 g/l yeast extract, 15 g/l bactoagar (Gibco) and 8 g/l NaCl (Riedel-deHaën)) containing 100 μg/ml ampicilline and 10 μg/ml tetracycline. Bacterial colonies were counted,scraped from the plates and used as inoculum for the next round of phage rescue according to Marks et al. (1991).

### Example 2

### Analysis of Isolated Phage

Monoclonal phages were isolated according to Marks et al. (1991) and isolated phages were tested for binding using flow cytometry analysis on Human Microvascular Endothelial cells, that were used for the selection procedure (HDMEC-1). For this, HDMEC-1 cells were detached from the flasks by incubating the cells in 50 mM EDTA in PBS at 4° C. for 15 min. Cells were then incubated with 100 μl pre-blocked phage maxiprep (50 μl phage prep was incubated for 15 min at 4° C. with 50 μl PBS 4% milk powder (Protifar) for 30 min at 4° C. The cells were then washed 4 times with 200 μl PBS 1% BSA and incubated for 30 min at 4° C. with Mouse anti M13 (Amersham) that was diluted 1:500 in PBS 1% BSA. After 4 times washing the cells were incubated with Goat anti Mouse PE 1:500 (Southern Biotechnology Associates, Inc) for 30 min at 4° C. After 4 times washing with PBS 1% BSA cells were analyzed on a FACS calibur for positive binding of the phages to the HDMEC cell line that was used for the selection procedure.

FIG. 1 shows the specific FACS staining of HDMEC-1 cells that were incubated with the isolated phage. The single phage that was isolated and that carries a scFv that recognizes HDMEC-1 cells specifically was named FibMab. The P9 scFv served as a positive control since it was identified in the same screen as FibMab for binding to the HDMEC-1 cells and the Thyro scFv (identical to the Tg scFv) served as a negative control (see below).

### Example 3

### Analysis of FibMab Using a Large Panel of Different Cell Lines

FibMab was analyzed on a large panel of cell lines and Peripheral Blood Leucocytes using the same staining protocol as described before. A positive score (depicted as +) was determined by a staining intensity that was higher than that of a negative phage staining. Results are shown in Table I.

TABLE I
Cells that were tested for binding by FibMab.

| cell line | score | cell line | score | cell line | score |
|---|---|---|---|---|---|
| Ls 174 T | − | RPMI 8226 | + | HDMEC-1 | + |
| Ls 180 | − | CEM | − | UM26 | − |
| UM 9 | − | HELA | − | HUVEC | − |
| UM 3 | − | HepGII | − | ECL-4 | − |
| Daudi | − | BHK | − | U266 | − |
| Raji | − | COS | − | DOX 40 | +/− |
| HL60 | − | K563 | − | HT29 | − |
| Fravel | − | THP1 | − | U937 | − |
| Jurkat | − | 293T | − | | |

The results depicted in Table I show that FibMab recognizes an epitope present on three cell lines: The formerly used cells HDMEC-1, the plasma cell line RPMI 8226 and the plasma cell line DOX 40, which gave a slightly less intense staining as compared to HDMEC-1 and RPMI 8226. DOX 40 is a doxorubicine resistant variant of the cell line RPMI 8226. FIG. 2 shows the FACS analysis of FibMab on the plasma cell line RPMI 8226.

FIGS. 3, 4 and 5 show the FACS analyses of FibMab on different compartments of the tested peripheral blood leucocytes (PBL's) as indicated in the short descriptions. Thyro (Tg) and P9 scFv's served as negative and positive controls respectively (see above).

### Example 4

### Determination of Heavy and Light Chain Variable Region Sequences Present in FibMab

The nucleotide sequences of the variable heavy chain region (Vh) and the variable light chain region (Vl) were determined using the ABI373 Xl system (Applied Biosystems) following the instructions given by the manufacturer. For this, 8 μl of big dye terminator mix, 3.2 pmol of primer (diluted in 1 μl), 5 μl plasmid miniprep (Qiagen) and 6 μl of bidest were mixed and incubated in a thermocycler (Rocidile III, Apligen). The PCR program that was used was: 96° C. 1 min followed by 25 times three steps: 1) 96° C. for 10 sec, 2) 50° C. for 5 sec and 60° C. for 4 min. The reactions were then cooled to 4° C. and analyzed by the ABI 373 XL system. From the nucleotide sequence, the amino acid sequence of the heavy (Vh) and light (Vl) chain and the synthetic hypervariable CDR3 region (binding domain of and antibody) were determined. Reagents from the Big Dye terminating kit (PE) were applied according to standard protocols well known to persons skilled in the art. Standard FDseq and M13R sequence primers were used. Identified sequences are given in Table II.

TABLE II

Classification and Amino acid sequences
of the scFV present on FibMab.
Subclass nomenclature for Vh and Vl
are depicted.

| | |
|---|---|
| Heavy chain | |
| Vh FibMab: | Vh3 1-3 DP30.7 |
| CDR3 region: | EDTAVYYCAR NPFQSS FDYWGQ |
| Light chain | |
| Vl FibMab: | Vk1 |
| CDR3 region: | EDFATYYC SQFSTMPGGFGQGTK VEIK |

### Example 5

### Immuno Histochemical Analysis of the scFv from Phage FibMab on Grawitz Tumor Cells and Healthy Kidney Tissue

For the preparation of scFv preps, the DNA encoding the scFv was cloned into a His-Myc construct by digesting the pPVT plasmid of FibMab with NcoI and NotI to excise the Vh and Vl of the scFv of FibMab and ligating this insert into a pPVT construct containing both a Histidine-taq and a Myc-taq for detection and purification, resulting in the construct FibMab-His/myc. This plasmid was deposited at the ECACC on 15 Jan. 2002, under accession number 02011518.

scFv (TES) preps were prepared using an osmotic shock to extract the scFv's from the periplasmic space of the bacteria E. coli SF110f′ according to Marks et al. (1991). After extraction, the scFv preps (TES preps) were then dialyzed three times at 4° C. against 5 liter of PBS for 4 hrs. Cryosections of a Human Grawitz tumor and a healthy part of the same kidney were placed on a Silan (Sigma) coated glass slide and dried overnight at room temperature. The cryosections were re-hydrated in PBS and blocked with PBS 4% BSA (ICN) for 20 min. The sections were then incubated with undiluted scFv preps, that were prepared as described supra or EN4 monoclonal antibody (Monosan) directed against CD31 (diluted 1:200) in PBS+1% BSA for 45 min as a positive control for endothelial cells staining. Then, sections were washed 3 times in PBS/0.05% Tween20 for 5 min and incubated with either culture medium from the 9E10 cell line (containing the anti Myc epitope antibody (Boehringer Mannheim) or a Rabbit Anti-Mouse antiserum (RAMPO, DAKO, diluted 1:250 in PBS+1% BSA) for 30 min. After 3 times washing in PBS/0.05% Tween20, the scFv stainings were incubated with RAMPO and the EN4 sections were incubated with a Swine Anti-Rabbit antiserum (SWARPO, DAKO, diluted 1:250 in PBS+1% BSA) for 30 min. Subsequently, the sections with the EN4 staining were washed 3 times with PBS/0.05% Tween20 for 5 min and 3 times with PBS for 5 min. The sections with the scFv staining were washed 3 times with PBS/0.05% Tween20 for 5 min and incubated with SWARPO for 30 min. After this SWARPO incubation the sections were again washed 3 times in PBS/0.05% Tween20 for 5 min and 3 times in PBS. The staining following these procedures was performed with DAB tablets dissolved in PBS (Sigma) and all sections were counter-stained with heamatoxiline (Merck) for 30 sec. The sections were imbedded using glycergel (DAKO) and analyzed using a light microscope.

Other tumor tissues that were analyzed using the same protocol as described for the healthy kidney tissue and the Gravitz tumor cells included: Breast Carcinoma, Prostate Carcinoma, Colon Carcinoma, Lung tumor, Melanoma cells, Pancreas tumor, Bladder tumor and Head and Neck tumor cells. Moreover, from the same tissues healthy cells were also tested for FibMab reactivity, except for healthy lung tissues. None of the healthy tissues showed staining with FibMab. FIG. 6A and B show immuno-histochemical staining of either scFv FibMab or an anti-CD31 scFv (EN4) on different human tumor tissues and healthy human tissues, which were:

#1-5 lungcarcinoma with FibMab (positive area)

#6 lungcarcinoma with FibMab (negative area)

#7-9 mamma carcinoma with FibMab

#10-11 melanoma with FibMab (positive area)

#12 melanoma with FibMab (negative area)

#13-15 Grawitz kidney tumor with FibMab

#16 healthy kidney tissue with FibMab

#17 transplantation kidney with FibMab

#18 transplantation kidney with anti-CD31

#19 healthy pancreas with FibMab

#20 pancreas tumor with FibMab

#21 bladder tumor with FibMab

#22-24 head and neck tumor with FibMab

#25 healthy colon with FibMab

#26 colon carcinoma with FibMab

#27 prostate tumor with FibMab

#28 healthy prostate with FibMab

In pictures numbers 1 to 5 of FIG. 6, a lung carcinoma is stained with scFv Fibmab. Clearly visible is the presence of separate positive (stained by Fibmab) and negative (not stained by Fibmab) areas in the tumor tissue. Picture 2 shows a distinct stromal staining in the tumor and no clear endothelial cell or vessel/capillary staining can be observed. Picture 3 shows a large area of tumor stroma that is negative for Fibmab, indicating that Fibmab only recognizes a portion of the tumor stroma in lung carcinoma tissue. Picture 4 and 5 show a positive area at a larger magnification. Pictures 7 and 8 show positive staining on a mamma carcinoma in which a vascular or capillary structure can be recognized but also a stromal staining is present. Picture 9 shows a negative area in the tumor tissue again indicating that only a part of the tumor is recognized by scFv Fibmab. In Pictures number 10, 11 and 12 a melanoma tumor is stained with scFv Fibmab and picture 12 shows a relatively “normal and healthy” part of the tumor that also is not recognized by scFv Fibmab. Again a clear stroma staining can be seen in this melanoma tissue. Pictures 13, 14 and 15 show a staining with scFv Fibmab on a Grawitz (kidney) tumor at different magnifications. Clearly visible is a vascular or capillary staining in this tissue. The tumor is highly vascularized. 16 and 17 are stainings with Fibmab on transplantation kidney and this is regarded as healthy kidney. No staining can be found in healthy kidney tissue. Picture 18 shows a positive endothelial staining with monoclonal antibody EN4 (recognizing CD31) on transplantation kidney and clearly visible are the vessels in the tubular area and positive staining of the capillaries in the glomerulus of the kidney. Picture 19 showed a healthy pancreas stained with scFv Fibmab and no staining can be seen whereas the pancreas tumor in picture 20 shows a clear stromal staining with scFv Fibmab. Picture 21 shows a positive staining on a bladder tumor in which capillary or vessel structures can be seen. Pictures 22 and 24 show a clear positive staining in a head and neck tumor with scFv Fibmab and picture 22 shows a relatively healthy portion of the same tissue which does not react with the scFv Fibmab. No staining with scFv Fibmab can be found in healthy colon (picture 25) but a colon carcinoma reacts strongly with the scFv Fibmab in the tumor stroma of this tissue. Picture 27 depicts a positive stromal and vessel like staining with scFv Fibmab in a prostate tumor and the healthy tissue control is negative for binding the scFv Fibmab.

In conclusion, scFv Fibmab does only react with all the tumor tissue tested sofar and does not show any reactivity with the healthy tissue controls. It appears that the scFv Fibmab recognizes either a stromal factor in all these tumors but also positive staining can be found in the vasculature in parts some of these tumors.

### Example 6

### The Use of FibMab in Immuno-Precipitation Experiments to Analyze the Recognized Antigen

scFv TES preps (prepared as described supra) from the His-Myc construct were used for immuno-precipitation. Immuno-precipitations were performed on the plasma cell line RPMI 8226, that stained positive for FibMab (Table I) on FACS analysis. Cells were cultured in RPMI 1640 medium+25 mM Hepes, L-glutamine and 5% Fetal Calf Serum (FCS). RPMI 8226 is a semi-adherent plasma cell line and cells do detach from the plates easily by shaking gently. B-cells were obtained from tonsil patient material and prepared according to Van der Vuurst de Vries et al. (1999a) and served as a negative control cell line in these experiments for FibMab. scFv's that were used were: FibMab and P9 (scFv's that recognizes endothelial cells but with a different expression pattern, isolated according to the same selection procedure) on RPMI 8226 cells, III-1 (twice) and I-1 (B-cell specific scFv's, Van der Vuurst de Vries et al. 1999b) were used as a positive control for immuno-precipitation procedures on tonsil B-cells (Van der Vuurst de Vries et al. 1999b), Tg and DNP are scFv's that were selected against peptides according to De Kruif et al. (1995) and served as negative controls on RPMI 8226 cells and as preclearing scFv respectively. III-1 was also used on RPMI 8226 cells where it served as a negative control for precipitated proteins. Pre-clearing scFv's were dialyzed against PBS as described supra. All scFv's were coupled to chelating sepharose beads (Amersham).

Preparation of Sepharose Beads

Sepharose beads were prepared and coupled to the scFv's as follows. 500 1μl Sepharose beads were washed 2 times with 1 ml bidest water, followed by 2 times with 1 ml 50 mM EDTA, 1 time with 2 ml 100 mM CoCl₂, 1 time with 2 ml bidest water and 2 times with 1 ml PBS. The beads were then incubated with 10 ml scFv prep by rocking for 30 min on a rocking platform and then washed two times with 4 ml PBS. These scFv bound beads were incubated with PBS+0.03% H₂O₂ while rocking for 2 hrs at room temperature and subsequently washed with 2 times 2 ml PBS. Beads were then washed once with 2 ml PBS+50 mM EDTA and washed two times with 2 ml PBS. Beads were then washed with 2 ml PBS+0.5 M Imidazole and again washed 2 times with 2 ml PBS and stored at 4° C. till further use, which was typically within 24 hrs.

Preparation of Cells

HDMEC-1 cells were grown as described supra. For each separate immuno-precipitation 2×10⁶ B-cells were used and all steps were performed at 4° C. Cells were washed 3 times with 20 ml PBS and subsequently resuspended in 1 ml PBS. 10 μl Normal Human Serum (NHS) sulpho-biotin (200 mg/ml in DMSO) was added to the cells and incubated for 30 min with occasionally gentle stirring. Then 10 μl 1 M NH₄Cl in PBS was added to the cells and washed twice with 2 ml 10 mM NH₄Cl. Cells were again washed twice with PBS and then lysed in 1 ml Lysis buffer (1% Triton X100) plus protease inhibitors (Cocktail tablets, Boehringer Mannheim).

Immunoprecipitations

The cell lysates were spun for 1 min at 14,000 rpm and pre-cleared 3 times for 1 hr at 4° C. with 100 μl beads coupled to scFv DNP. These pre-cleared lysates were divided over the different immuno-precipitations and incubated with 40 μl beads each at 4° C. while rocking overnight. Then, the beads were washed 10 times for 10 min with lysis and 10 times 10 min with PBS. Beads were resuspended in 40 μl PBS and 4 μp 10X Laemmli sample buffer was added. Samples were boiled for 1 min and separated using a 7%-SDS/PAGE gel. Separated proteins were then blotted onto a PVDF membrane (Boehringer Mannheim) and blocked overnight in 2% gelatin. The membrane was then incubated for 30 min with streptavidin HRP (Amersham) and washed 10 times for 5 min in PBS/0.1% Tween20 and washed 3 times for 5 min with PBS. For development of the blot Chemoluminescence blotting substrate (Boehringer Mannheim) was used following the instructions of the manufacturer and Hyperfilm (Hyperfilm MP) was used for detection of the produced signal. FIG. 7 Shows the blot of the SDS/Page separated immuno-precipitations of the different scFv's on tonsil B-cells and the plasma cell line RPMI 8226. Bound biotinylated membrane proteins were visualized using streptavidine HRP in combination with Supersignal (Pierce). The result shows the positive signal on tonsil B-cells for scFv's III-1 and I-1 as was expected. scFv FibMab shows a specific band of a high molecular weight and P9 does not precipitate any proteins at the used conditions. Controls I-1 and Tg scored negative on tonsil B cells and RPMI 8226 cells respectively, as expected.

Protein Sequencing

To obtain sufficient amounts of antigen that is recognized by FibMab the whole procedure was scaled up 10 times and the specific FibMab IP band was excised from the SDS/PAGE gel and stored on ice. This was send to TOPLAB (Hamburg, Germany) where some amino acid sequences within the recognized antigen were determined. Three peptide stretches that were sequenced were identified as being a fragment of the fibronectin protein. These identified peptides were:

1) AFTDVDVD

2) IPGHLNSYTIK

3) SSPVTGYRVT

After analysis using the NCBI database, it was found that all peptide tags showed homology with the fibronectin precursor, which is the unspliced fibronectin. Peptide tag 1) showed homology with the ED-A fibronectin splice variant.

### Example 7

### Cloning Procedures to Obtain a Fully Human IgG1 Monoclonal Antibody from the FibMab scFv

The procedures that were followed for the cloning of fully human IgG1 monoclonal antibodies were described by Boel et al. (2000).

Primers

| | | |
|---|---|---|
| 1. | HAVT + HindIII: | 5′-AAG CTT CCC ATA AGG AAT TCG GAT CCC |
| | | ACG ATG GCA TGC CCT GG-3′ |
| 2. | M13: | 5′-GT AAA ACG ACG GCC AGT-3′ |
| 3. | Vκ1a: | 5′-TGT GAC ATC GAG CTC ACC CAG TCT CCA TCC-3′ |
| 4. | Jκpan: | 5′-TTC TCG ACT TGC GGC CGC AAA GTG CAC TTA CGT |
| | | TTG ATC TCC ACC TTG GTC CC-3′ |

Cloning of the Vh Fragment

The Vh fragment was excised from the pPVT vector containing the genes encoding the scFv FibMab with NcoI and XhoI restriction enzymes, purified over gel and ligated into pLeader, which is a vector that contains a Kozak sequence and a donor splice site that was digested with NcoI and SalI and purified over gel. This resulted in a plasmid named pLeader VhFibMab. The cloned insert was subsequently amplified by PCR: 96° C. for 5 min followed by 28 times 1)96° C. for 30 sec, 2) 58° C. for 30 sec, 3) 72° C. for 1 min and finalized by 72° C. for 7 min. The reaction product was then cooled to 4° C. Primers that were used for the PCR were M13 and “HAVT+HindIII” for introduction of a HindIII site at the 5′-end. Furthermore, the HAVT20 leader sequence was altered slightly by changing the translation start sequence into 5′-CCACGATGG-3′. pcDNA 3.1 ZEO (Promega) was mutated by eliminating the NotI in the multiple cloning site (pcDNA 3.1 δNotI ZEO) because the NotI could interfere with cloning further downstream. This mutation was introduced by digesting pcDNA ZEO with NotI and subsequently by filling in the sticky ends using Klenow enzyme and free nucleotides. The constant heavy region of a human IgG (Cγl) fragment with the Vh of another scFv (UBS-54 directed against human Ep-Cam) was inserted using the BamHI and EcoRV restriction sites. The PCR product of the Vh FibMab was then swapped with HindIII and NotI into pcDNA 3.1 ZEO δNotI+Cγ1. FIG. 8B shows the schematic representation of the cloning procedures described for Vh. The resulting plasmid designated pcDNA+Vh Fibmab Cg1, was deposited at the ECACC on 15 Jan. 2002, under accession number 02011519.

Cloning of the Vl Fragment

The Vl fragment was amplified by PCR by using the same PCR protocol as described in the cloning of the Vh fragment from the pPVT vector with primers “Vκ1a” and “Jκpan”. In this step, the NotI site was deleted and reintroduced at the 3′ site from the donor splice site, which was also introduced by primer Jκpan. The PCR product was digested with SacI and NotI and cloned into the pLeader vector which is a vector that contains a Kozak sequence and a donor splice site with SacI and NotI resulting in pLeader VlFibMab. This fragment was again amplified with primers “HAVT+HindIII” and “M13” for the same purpose as in the Vh cloning to introduction a HindIII site at the 5′-end. Furthermore, the HAVT20 leader sequence was altered slightly by changing the translation start sequence into 5′-CCACGATGG-3′._The Vl of a different scFv (UBS-54) with the constant region of the light chain of a IgG1 (Cκ) was cloned into pcDNA 3.1 δNotI ZEO using BamH1 and EcoR1. The Vl of FibMab was then swapped using HindIII and NotI. FIG. 8A shows the schematic representation of the cloning procedures described for Vl. The resulting plasmid designated pcDNA+Vl Fibmab Ck, was deposited at the ECACC on 15 Jan. 2002, under accession number 02011520.

The same procedure is followed when cloning the scFv FibMab into vectors that can produce human IgG2, IgG3, IgG4, IgA1, IgA2, IgM and IgE. These different antibodies and possible fusion proteins are used in in vivo, ex-vivo and in vitro tumor models to test their capacity to diagnose and/or block or inhibit angiogenesis, matrix formation and reduction or elimination of tumors.

### Example 8

### Characterization of the FibMab Epitope

Binding of FibMab to Fibronectin Directly Coated to Plastic.

To check Fibmab binding to fibronectin, purified plasma fibronectin was coated directly to a maxisorp plate at a concentration of 100 μg/ml in a serial dilution of ⅓ for 1 hour. The last lane was left out of the serial dilution and was incubated with PBS. The plate was then blocked with PBS+4% BSA for 1 hour at room temperature. Either scFv preps (undiluted) or a monoclonal antibody (FN30-8, 1:1000) were added to the plates with the fibronectin for 1 hour at room temperature. The plate was then washed 6 times with PBS 0,1% tween 20 and the scFv wells were incubated with 9E10 sub. The monoclonal antibody staining was incubated with PBS 1% BSA. The plate was again washed with PBS 0,1% Tween 20. For detection of scFv's and monoclonal antibody, a Rabbit anti Mouse HRP (RAMPO 1:1000, DAKO) was used. The substrate used for the HRP was ABTS (Boehringer Mannheim). The resulting extinction was measured at 405 nm. The result is shown in FIG. 9. Only a very weak binding of Fibmab to the plastic-coated fibronectin was detected compared to the positive control (FN30-8) used in this assay.

Gelatin/Collagen Binding of Purified Fibronectin.

The sequence data implied that the ligand for Fibmab was fibronectin. However, in the ELISA described supra we could not show a strong binding of Fibmab to plastic coated purified plasma-derived fibronectin in ELISA. The fact that Fibmab was generated on the cell line HDMEC, suggested that we are dealing with a cell-associated form of fibronectin. This form is present in extracellular matrix where it forms an insoluble network by interaction with other matrix components such as collagens and proteoglycans, thus assisting cell migration and the maintenance of tissue integrity (reviewed by Magnusson and Mosher, 1998). To mimic matrix-associated fibronectin, fibronectin was immobilized through the binding to pre-coated collagen and bovine gelatin (cell culture grade). Gelatin is a degraded form of collagens and is commonly used as substitute for different forms of collagens. Furthermore, the cell line HDMEC used to generate this antibody was cultured on gelatin. A maxisorp plate was coated with either 100 μg/ml gelatin (Merck) or 100 μg/ml Collagen (purified collagen for cell culture, Vitrogen) in PBS for 2 hours at room temperature (100 μl/well). The plate was blocked with PBS+4% BSA for 1 hour. Fibronectin was added to the plate at a starting concentration of 75 μg/ml in a serial dilution and incubated in the well for 1 hour at room temperature. The last well was not included in the serial dilution and did not contain any fibronectin. After incubation each lane was incubated with scfv prep or a monoclonal antibody against fibronectin. The plate was washed 6 times with PBS 0,1% tween 20 and the scFv lanes were incubated with 9E10 sup (contains anti-Myc monoclonal antibody, Boehringer Mannheim) for 45 minutes at room temperature. The lane with the monoclonal antibody was incubated with PBS+1% BSA. The plate was then washed again 6 times with PBS+0,1% tween 20 and all wells were incubated with Rabbit anti Mouse HRP for 45 minutes at room temperature. The monoclonal antibody FN9-1 was used as a positive control to show that there was no difference in the amount of fibronectin present in the different ELISA's.

Results are shown in FIG. 10.

FIG. 10A shows that in all cases (fibronectin directly coated or bound through gelatin or collagen) equal amounts of fibronectin was immobilized to the wells. FIG. 10B shows that Fibmab hardly recognized directly coated fibronectin, whereas gelatin-bound fibronectin showed a strong increase/induction of Fibmab epitope expression or exposure. Binding of fibronectin to pre-coated collagen only slightly induced expression of the Fibmab epitope. Such titration experiments can be used to determine the factor by which binding of FibMab to fibronectin is enhanced by the interaction with gelatin. In the present example, said factor is higher than twenty when comparing fibronectin directly bound to plastic with fibronectin bound via gelatin.

FIG. 10B showed that direct binding by purified plasma-derived fibronectin induced some Fibmab epitope expression. To circumvent a possible artificial conformational change of fibronectin due to plastic binding, a sandwich ELISA was performed as follows. A polyclonal anti-fibronectin was used as capture antibody (Capel), then purified plasma fibronectin was added and binding of fibronectin was detected with a monoclonal antibody (FN30-8), followed by RAMPO and the PO-substrate ABTS. Recognition of gelatin-bound fibronectin was measured simultaneously. FIG. 11A shows that immobilization of plasma-derived purified fibronectin by-a polyclonal antibody, which keeps the fibronectin in its most native conformation, showed no expression of the Fibmab epitope. However, when the same fibronectin batch was immobilized through gelatin-binding, a very pronounced expression of the Fibmab epitope was observed (FIG. 11B). These data indicate that Fibmab epitope expression is absent in soluble fibronectin, slighty induced in plastic-bound and collagen-bound fibronectin, but very pronounced in gelatin-bound fibronectin.

To exclude the possibility that the expression of this Fibmab epitope was due to the purification procedure, human serum and plasma (diluted 1:3 to obtain a similar fibronectin concentration used in the experiments described supra) were used as fibronectin source in a “gelatin sandwich” ELISA, with essentially the same results as described supra, demonstating that Fibmab epitope expression is not induced during purification of fibronectin from plasma.

Mapping the Localization of the FibMab Epitope.

To map the localization of the Fibmab epitope we tested fragments of fibronectin (provided by D. Mosher). The localization of the fragments (which were obtained by proteolytic cleavage; Chernousov et al, 1991) in the full length fibronectin is depicted in FIG. 12. The fragments (10 μg/ml) and full length fibronectin were directly coated to plastic and binding of Fibmab or control scFv (Thyro) was measured similar as described supra for the ELISA's. The total amount of immobilized fragments was determined with a polyclonal antibody (Poab) directed against fibronectin.

Binding of the antibodies was detected with the appropriate second antibodies (for scFv anti-myc monoclonal antibody followed by RAMPO and for the polyclonal antibody SWARPO, both purchased from DAKO).

The results are shown in FIG. 13. Like the full length fibronectin, the 40 and 70 kDa fragments showed moderate binding of Fibmab, whereas the 27 and 160 kDa showed no binding at all. These data show that the smallest fragment containing the Fibmab epitope is localized on the gelatin binding segment. To verify this result, higher amounts (50 μg/ml) of the 40- and 70 kDa fragment were coated directly to plastic in triplicate wells and binding of a concentration range of Fibmab scfv or control scfv (Thyro) was tested as described supra. Binding of scfv Fibmab was significantly higher to both fragments as compared to the control scfv Thyro (FIG. 14). These data confirm that the 40 kDa fragment of fibronectin, comprising the gelatin binding domain, at least in part contains the epitope for Fibmab.

The data described above demonstrate that FibMab has characteristics that define it as a new antibody compared to the antibodies that also bind to fibronectin known from the prior art. The fact that Fibmab recognized plasma-derived fibronectin once bound to gelatin, indicates that Fibmab does not interact with the ED-A or ED-B domain, since these domains are not expressed in plasma-fibronectin. This makes Fibmab a different antibody than the ones known from the prior art that also exhibit tumor-specific tissue distribution like L19 (Birchler et al, 1999), BC1 (Midulla et al, 2000), IST4 (van Vliet et al, 2001), IST-6 (Kaczmarek et al, 1994), all against ED-B or IST-9 against ED-A (Scarpino et al, 1999). FibMab recognizes a conformational epitope on fibronectin. The prior art discloses an anti-fibronectin antibody called FDC-6 (U.S. Pat. No. 4,894,326; U.S. Pat. No. 5,243,029; Matsuura et al, 1985; Matsuura et al, 1988, Mandel et al, 1995), which also may recognize a conformational epitope, but this epitope is a cancer-associated de novo glycosylation site in the C-terminal region of FN (IIICS), and hence the FDC-6 epitope differs from the epitope recognized by FibMAb. The only antibody that recognizes an epitope in the same region as Fibmab known in the prior art is L8, but contrary to FibMab, binding of the L8 antibody is strongly diminished when fibronectin is bound to gelatin (Chernousov et al. 1991), demonstrating that the antibodies and their epitopes are not identical.

### SHORT DESCRIPTION OF THE FIGURES

FIG. 1. FACS-staining of HDMEC-1 cells incubated with FibMab. The Thyro scFv is the same as Tg in example 5, identified by De Kruif et al. (1995) and served as a negative control. P9 is a scFv that was identified along with the FibMab scFv, which served as a positive control for staining.

FIG. 2. FACS-staining of FibMab on RPMI 8226 cells.

FIG. 3. FACS-staining of FibMab on Peripheral Blood Leucocytes (PBL's). Identification of B-cells by double staining PBL's with mouse anti CD20 (CLB, Amsterdam) to identify the B-cell population

FIG. 4. FACS-staining of FibMab on PBL's on the monocyte compartment.

FIG. 5. FACS-staining of FibMab on PBL's on the granulocyte compartment.

FIG. 6A. Immuno-histochemical staining of scFv FibMab on different human tumor tissues and healthy human tissues (see text for details).

FIG. 6B. Immuno-histochemical staining of scFv FibMab on different human tumor tissues and healthy human tissues (see text for details).

FIG. 7. Proteins immunoprecipitated by the FibMab scFv and a number of control scFv's on RPMI 8226 cells and tonsil B cells as indicated in the text. The molecular weight of the proteins (MW) is given on the right. The arrow points to the high molecular weight band that is immuno-precipitated specifically by the FibMab scFv.

FIG. 8A. Schematic representation of the cloning of the Vl FibMab vector.

FIG. 8B. Schematic representation of the cloning of the Vh FibMab vector.

FIG. 9. Direct ELISA on fibronectin.

FIG. 10. ELISAs with fibronectin bound through gelatin or collagen.

FIG. 11. Sandwich ELISA.

FIG. 12. Fibronectin and fragments thereof used for mapping the localization of the epitope.

FIG. 13. Mapping of the FibMab epitope on fibronectin fragments.

FIG. 14. Binding of FibMab dilutions to the 40 kDa and 70 kDa fibronectin fragments.

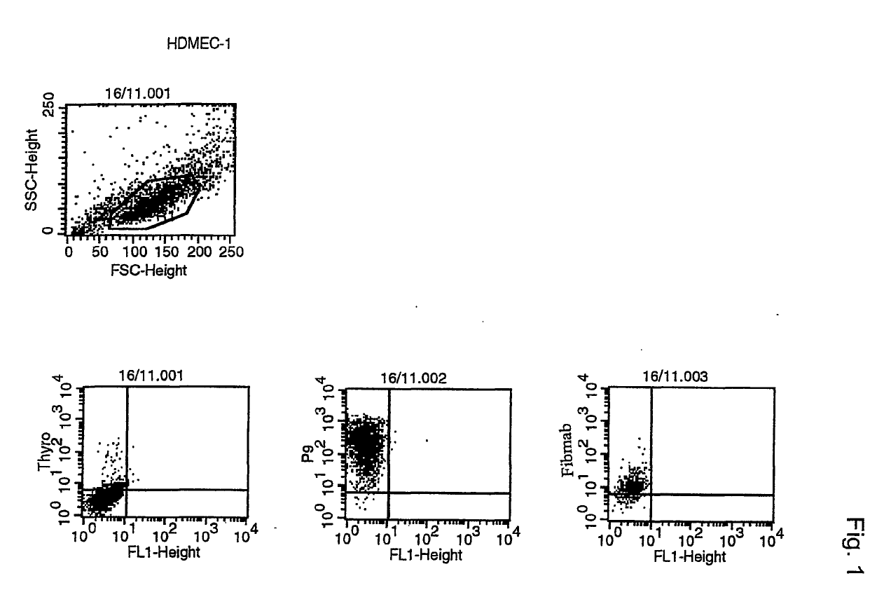

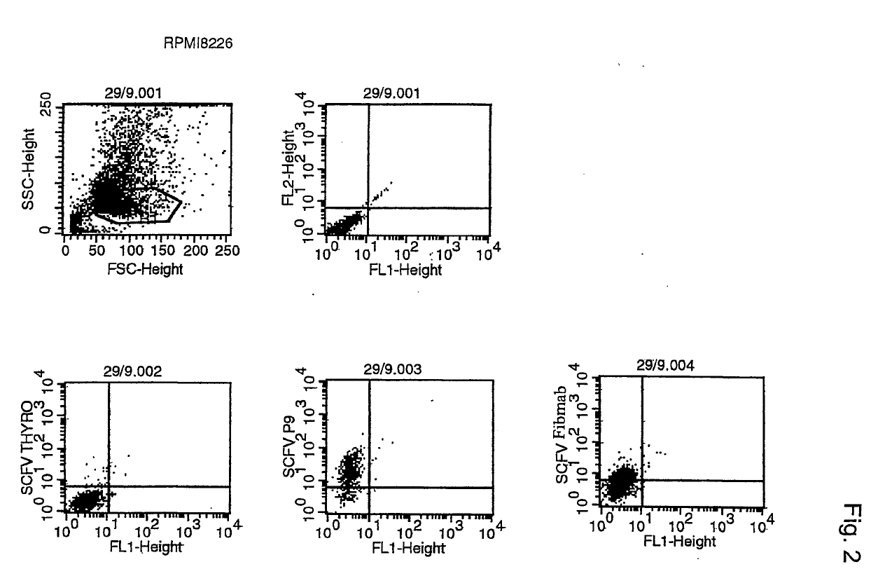

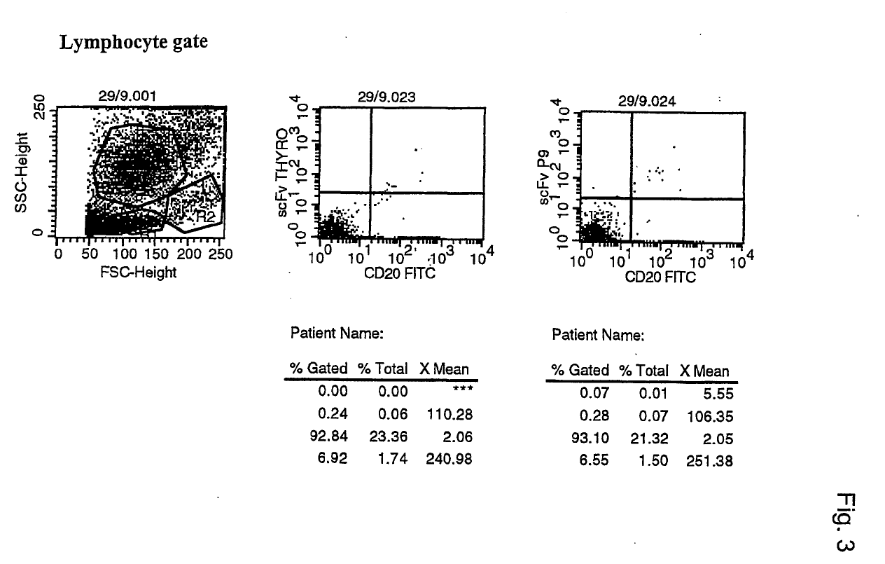

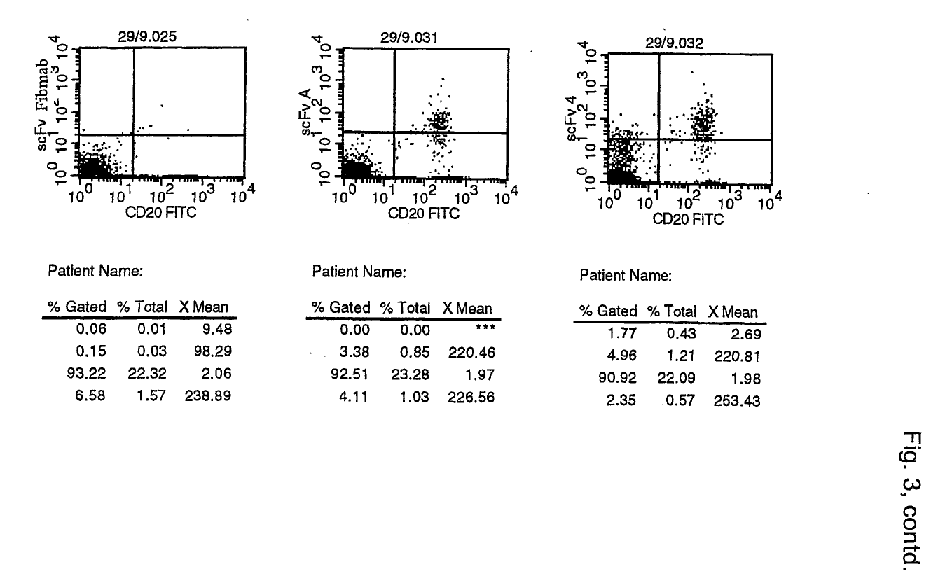

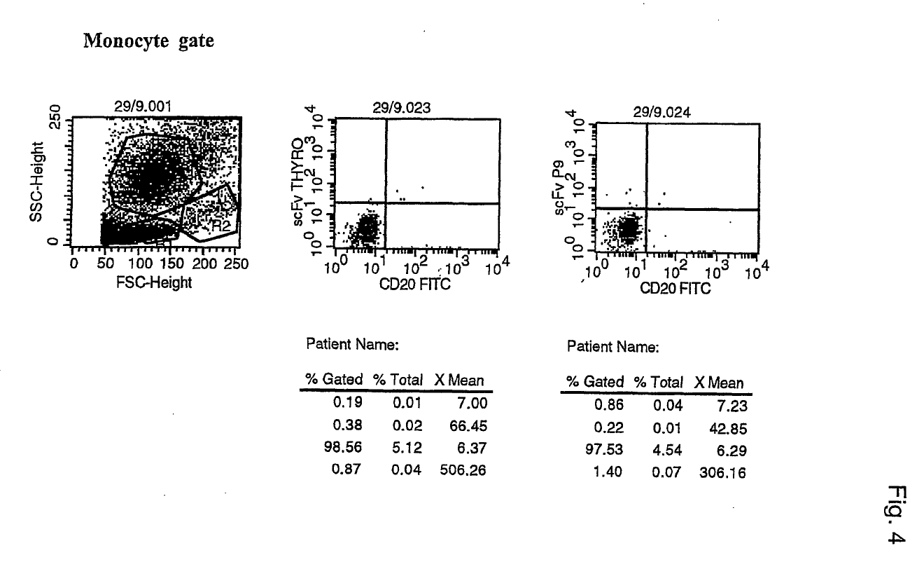

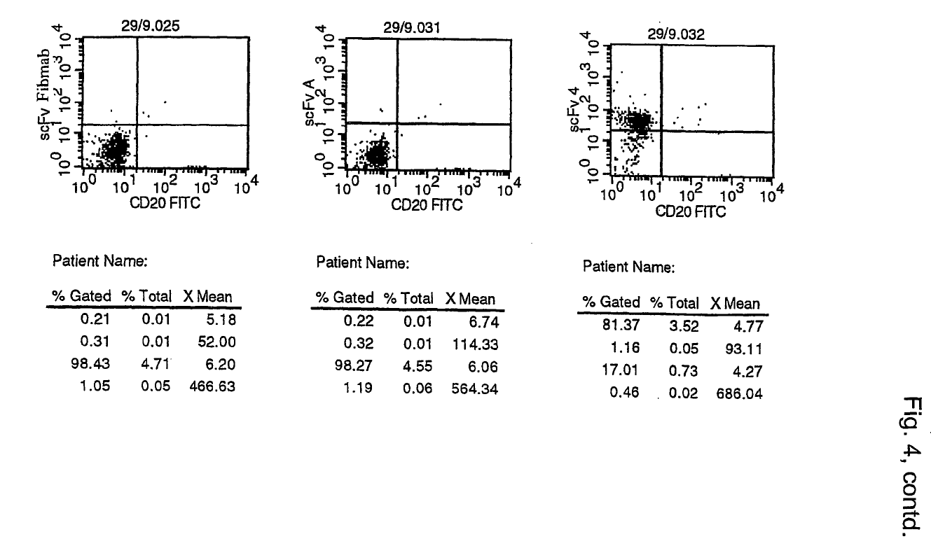

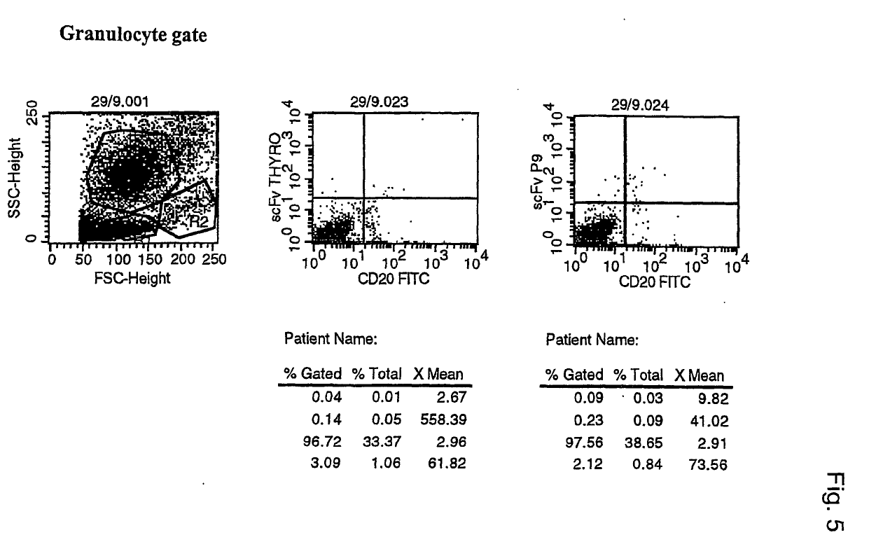

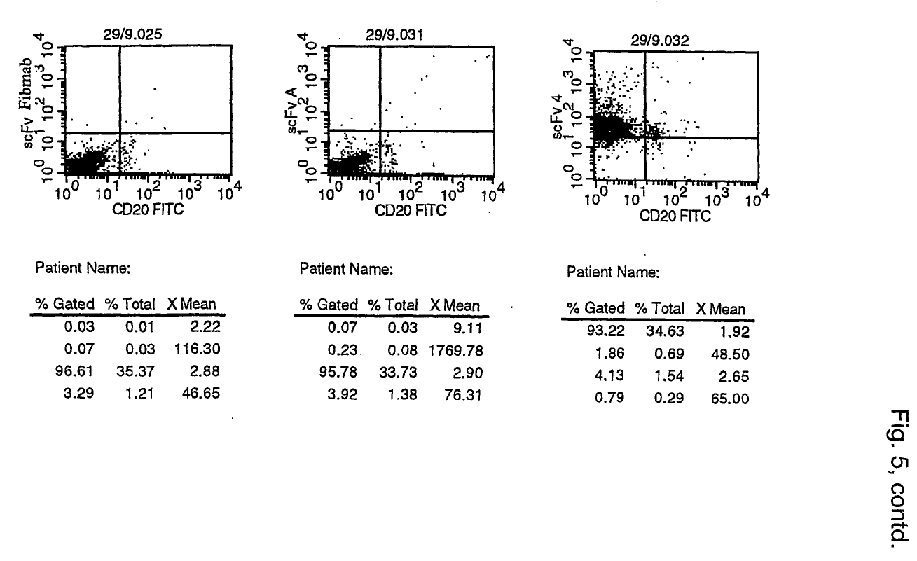

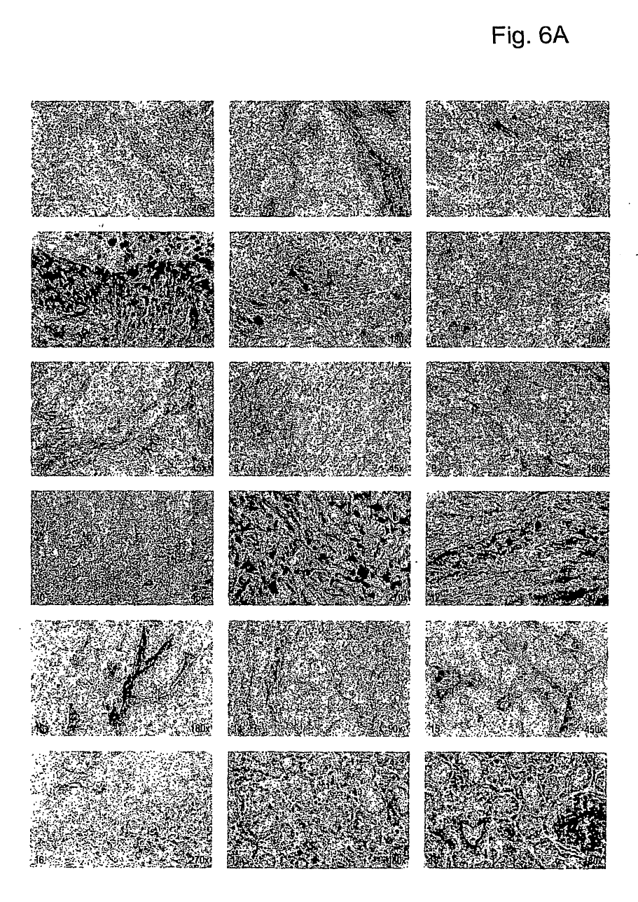

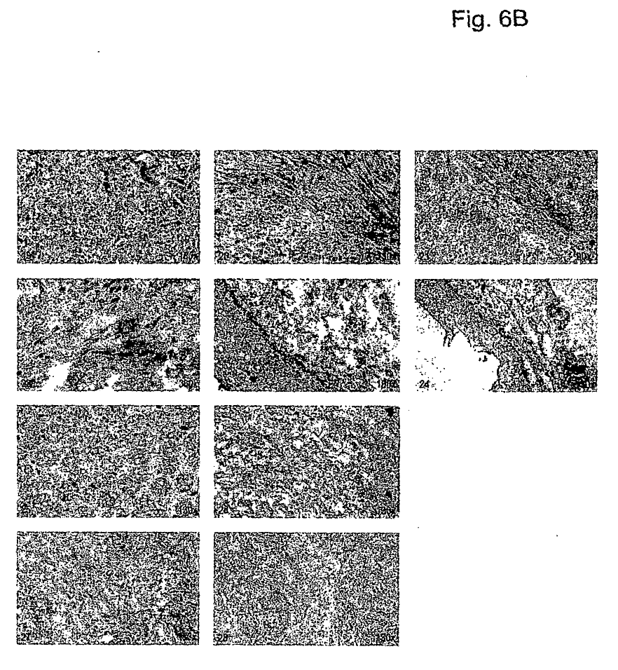

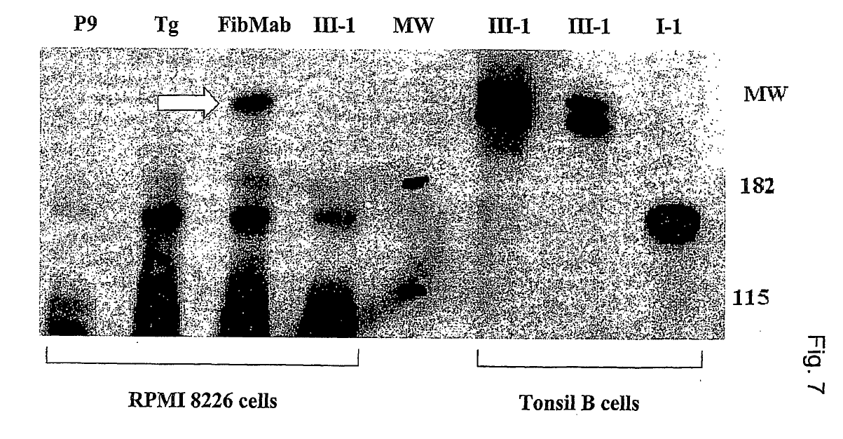

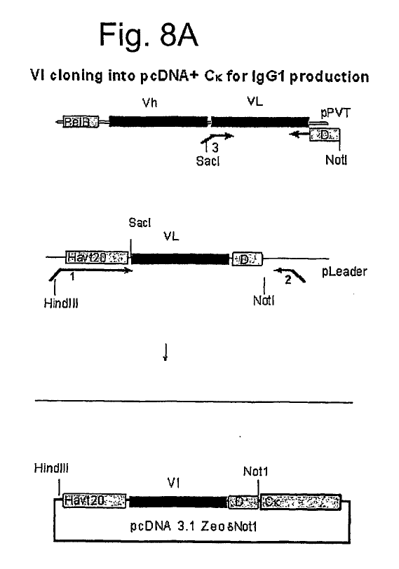

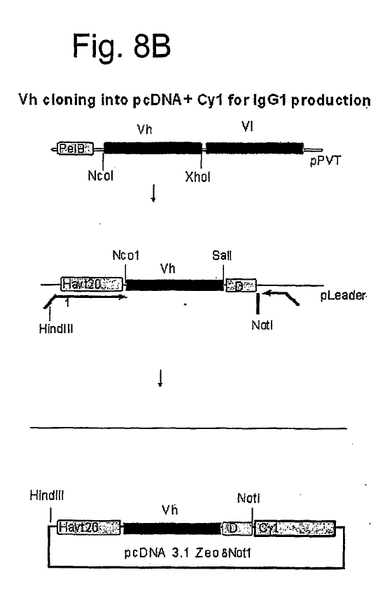

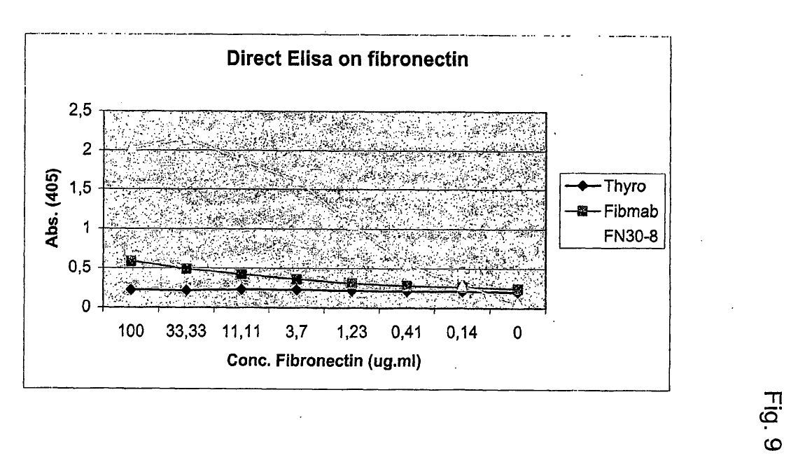

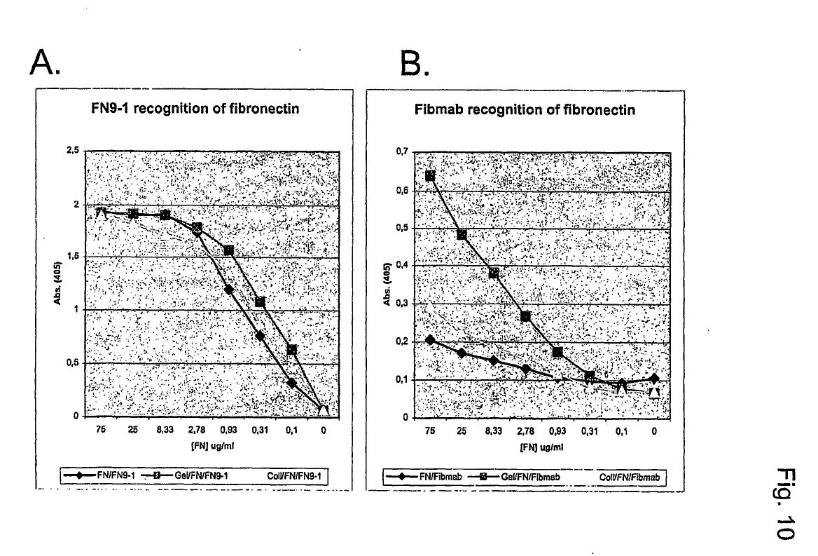

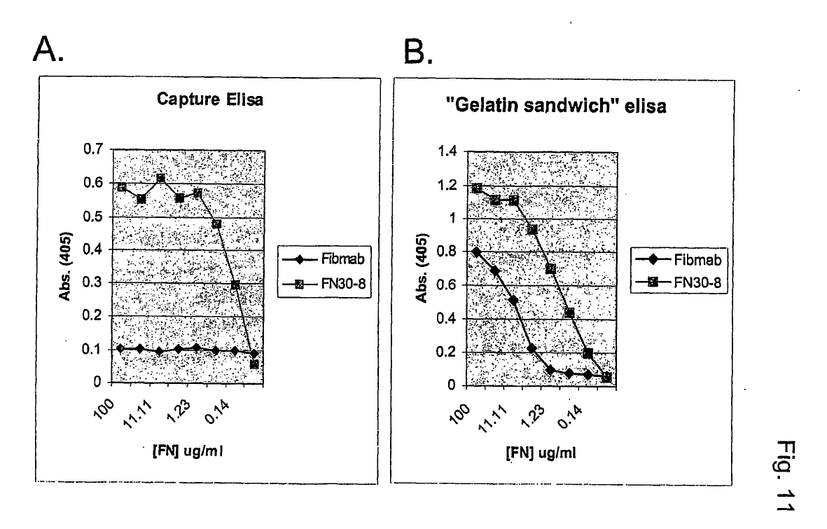

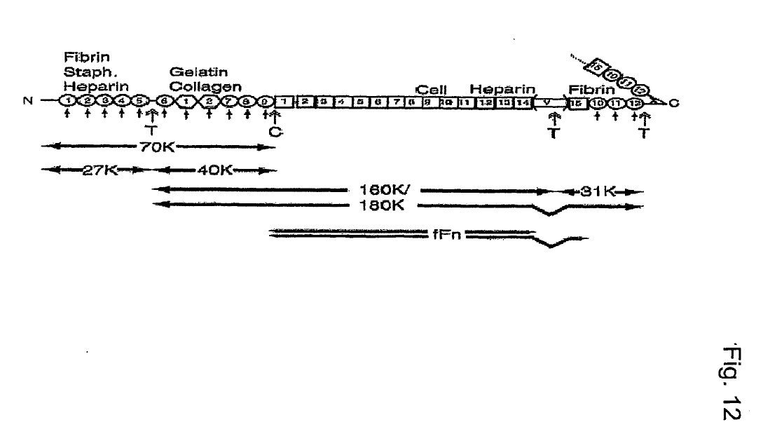

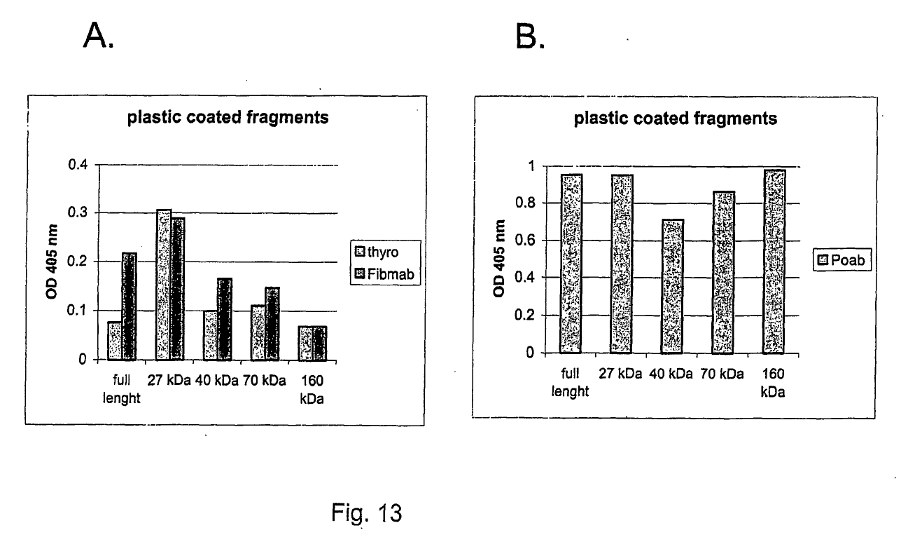

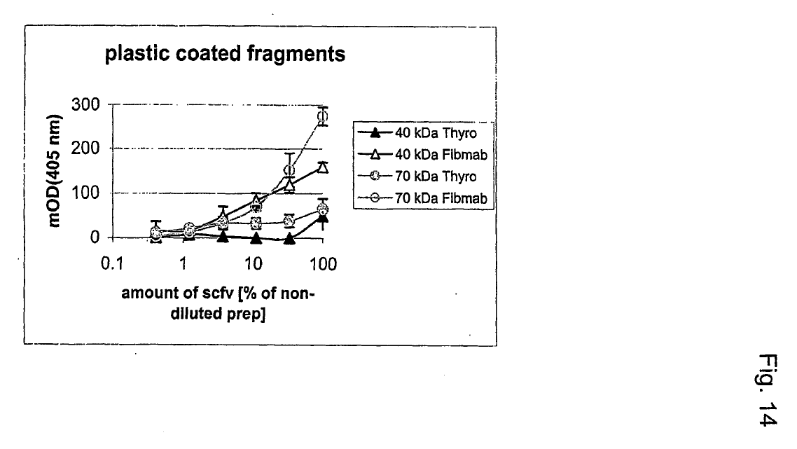

### REFERENCES

Ades E W,.Candel F J, Swerlick R A, George V G, Summer S, Bosse D C, Lawley T J. 1992. HDMEC-1: establishment of an immotalized human microvascular endothelial cell line. J. Invest. Dermatol. 99:683-690

Boel E, Verlaan S, Poppelier M J, Westerdaal N A, Van Strijp J A, Logtenberg T. (2000) Functional human monoclonal antibodies of all isotypes constructed from phage display library-derived single-chain Fv antibody fragments. J Immunol Methods. 26;239: 153-166

Birchler M, Viti F, Zardi L, Spiess B, Neri D. Selective targeting and photocoagulation of ocular angiogenesis mediated by a phage-derived human antibody fragment. Nat Biotechnol. October 1999; 17(10):984-8.

Chernousov, M. A., Fogerty, F. J., Koteliansky, V. E., Mosher, D. F. Role of the I-9 and III-1 modules of fibronectin in formation of an extracellular fibronectin matrix. 1991, J.Biol.Chem. 266(7): 10851-10858.

De Kruif J, Boel E, Logtenberg T. (1995) Selection and application of human single chain Fv antibody fragments from a semi-synthetic phage antibody display library with designed CDR3 regions. J Mol Biol 248:97-105

Kaczmarek J, Castellani P, Nicolo G, Spina B, Allemanni G, Zardi L. Distribution of oncofetal fibronectin isoforms in normal, hyperplastic and neoplastic human breast tissues. Int J Cancer. Oct. 1, 1994;59(1):11-6.

Liotta L A, Steeg P S, Stetler-Stevenson W G. (1991) Cancer metastasis and angiogenesis: an imbalance of positive and negative regulation. Cell 64:327-336

Magnusson M K, Mosher D F. 1998. Fibronectin: structure, assembly, and cardiovascular implications. Arterioscler Thromb Vasc Biol. 18:1363-1370.

Mandel U, Hamilton Therkildsen M, Reibel J, Sweeney B, Matsuura H, Hakomori S, Dabelsteen E, Clausen H. 1992. Cancer-associated changes in glycosylation of fibronectin. Immunohistological localization of oncofetal fibronectin defined by monoclonal antibodies. APMIS 100: 817-826.

Marks J D, Hoogenboom H R, Bonnert T P, McCafferty J, Griffiths A D, Winter G. 1991. By-passing immunization. Human antibodies from V-gene libraries displayed on phage. J. Mol. Biol. 222:581-597

Matsuura H, Hakomori S. 1985. The oncofetal domain of fibronectin defined by monoclonal antibody FDC-6: Its presence in fibronectins from fetal and tumor tissues and its absence in those from normal adult tissues and plasma. Proc. Natl. Acad. Sci. USA 82: 6517-6521.

Matsuura H, Takio K, Titani K, Greene T, Levery S B, Salyan M E K, Hakomori S. 1988. The oncofetal structure of human fibronectin defined by monoclonal antibody FDC-6. J. Biol. Chem. 263: 3314-3322.

Midulla M, Verma R, Pignatelli M, Ritter M A, Courtenay-Luck N S, George A J. Source of oncofetal ED-B-containing fibronectin: implications of production by both tumor and endothelial cells. Cancer Res. Jan. 1, 2000;60(1):164-9.

Pickford A R, Smith S P, Staunton D, Boyd J, Campbell I D. The hairpin structure of the (6)F1(1)F2(2)F2 fragment from human fibronectin enhances gelatin binding. EMBO J. Apr. 2, 2001;20(7) :1519-29.

Pujuguet P, Hammann A, Moutet M, Samuel, J L, Martin F, Martin M. 1996. Expression of fibronectin ED-A+ and ED-B+ isoforms by human and experimental colorectal cancer. American J. Pathology. 148:579-592

Reza Farnoud M, Farahnaz Farhadian, Samuel J L, Derome P, Peillon F, Yuan Li J (1995) Fibronectin isoforms are differentially expressed in normal and adenomatous human anteriar pituitaries. Int. J. Cancer. 61:27-34

Scarpino S, Stoppacciaro A, Pellegrini C, Marzullo A, Zardi L, Tartaglia F, Viale G, Ruco L P. Expression of EDA/EDB isoforms of fibronectin in papillary carcinoma of the thyroid. J Pathol. June 1999;188(2):163-7.

Van der Vuurst de Vries A-R, Clevers H, Logtenberg T, Meyaard L. (1999a). Leucocyte associated immunoglobulin-like receptor-1 (lair-1) is differentially expressed during human B-cell differentiation and inhibits B-cell receptor-mediated signaling. Eur. J. Immunol.29(10):3160-3167

Van der Vuurst de Vries A-R, Logtenberg T. (1999b). Dissecting the human peripheral B-cell compartment with phage display-derived antibodies. Immunology 98(1):55-62

Van Vliet A, Baelde H J, Vleming L J, de Heer E, Bruijn J A. Distribution of fibronectin isoforms in human renal disease. J Pathol. February 2001;193(2):256-262.

Werb Z. (1997) ECM and cell surface proteolysis: regulating cellular ecology. Cell 91:439-442

1. A proteinaceous molecule comprising a proteinaceous molecule capable of binding to fibronectin upon the interaction between fibronectin and one or more of gelatin, fragmented collagen and denatured collagen.

2. A proteinaceous molecule comprising a proteinaceous molecule molecule capable of binding to fibronectin, wherein said binding to fibronectin is enhanced by the interaction between fibronectin and gelatin, fragmented collagen or denatured collagen.

3. The proteinaceous molecule of claim 2, wherein said binding is enhanced by a factor of at least two.

4. The proteinaceous molecule of claim 3, wherein said binding is enhanced by a factor of at least twenty.

5. The proteinaceous molecule of claim 1, wherein said proteinaceous molecule binds an epitope that is induced by the interaction between fibronectin and one or more of gelatin, fragmented collagen and denatured collagen.

6. The proteinaceous molecule of claim 5, wherein said epitope is tumor-specific.

7. The proteinaceous molecule of claim 5, wherein said epitope coincides with angiogenesis.

8. A proteinaceous molecule comprising a proteinaceous molecule capable of binding to a matricryptic epitope in fibronectin.

9. The proteinaceous molecule of claim 8, wherein said matricryptic epitope is induced upon interaction of fibronectin with one or more of gelatin, fragmented collagen or denatured collagen.

10. The proteinaceous molecule of claim 8, wherein said matricryptic epitope is tumor-specific.

11. The proteinaceous molecule of claim 5, wherein the epitope is at least in part in the gelatin-binding domain of fibronectin.

12. The proteinaceous molecule of claim 1, wherein said fibronectin is part of an extracellular matrix.

13. The proteinaceous molecule of claim 12, wherein said extracellular matrix is in the vicinity of a tumor cell.

14. The proteinaceous molecule of claim 1, wherein said molecule is capable of binding to a neoplastic cell.

15. The proteinaceous molecule of claim 14, wherein said neoplastic cell is derived from a breast cell, prostate cell, colon cell, lung cell, skin cell, pancreas cell, bladder cell, head cell or a neck cell.

16. The proteinaceous molecule of claim 1, wherein said proteinaceous molecule is capable of binding to fibronectin in a microvessel.

17. The proteinaceous molecule of claim 1, wherein said proteinaceous molecule is capable of binding to a microvascular endothelial cell.

18. The proteinaceous molecule of claim 1, wherein said proteinaceous molecule is an antibody or a functional part or derivative thereof.

19. The proteinaceous molecule of claim 1, wherein said proteinaceous molecule is a single chain Fv fragment.

20. The proteinaceous molecule of claim 2, wherein said proteinaceous molecule is obtainable by expression from a plasmid designated Fibmab-His/myc, deposited at the ECACC under accession number 02011518.

21. The proteinaceous molecule of claim 18, wherein said antibody is human or humanized.

22. The proteinaceous molecule of claim 2, wherein said proteinaceous molecule further comprises a tag.

23. The proteinaceous molecule of claim 22, wherein said tag comprises a toxin and/or a radioactive substance.

24. The proteinaceous molecule of claim 1, comprising a heavy chain having a CDR3 region, said CDR3 region comprising the sequence EDTAVYYCAR NPFQSS FDYWGQ, as set forth in SEQ ID NO:1, or a derivative and/or functional analogue thereof.

25. The proteinaceous molecule of claim 1, comprising a light chain having a CDR3 region, said CDR3 region comprising the sequence EDFATYYC SQFSTMPGGFGQGTK VEIK, as set forth in SEQ ID NO:2, or a derivative and/or functional analogue thereof.

26. A method for the treatment or diagnosis of an individual suffering from or at risk of suffering from a disease, comprising administering to an individual or a sample, a proteinaceous molecule capable of binding to fibronectin upon the interaction between fibronectin and one or more of gelatin, fragmented collagen and denatured collagen, thereby treating or diagnosing a disease.

27. The method according to claim 26, wherein said disease is a neoplastic disease.

28. The proteinaceous molecule of claim 1, wherein said proteinaceous molecule is a medicament.

29. The proteinaceous molecule of claim 28, wherein said medicament is capable of treating a neoplastic disease.

30. The method according to claim 26, further comprising determining whether a cell is capable of specifically binding said proteinaceous molecule.

31. The method according to claim 30, wherein an epitope expressed on a subset of fibronectin expressing cells is a marker for a neoplastic cell and wherein said epitope is a tumor-specific matricryptic epitope.

32. A nucleic acid encoding a proteinaceous molecule comprising a nucleic acid sequence encoding a proteinaceous molecule or a part thereof, wherein said proteinaceous molecule, or part thereof, is capable of binding to fibronectin upon the interaction between fibronectin and one or more of gelatin, fragmented collagen or denatured collagen.

33. A cell comprising a nucleic acid according to claim 32.

34. The cell of claim 33, wherein said cell is a primate, rodent, bird or plant cell.

35. The cell of claim 33, wherein said cell is a human cell.

36. The cell of claim 33, wherein said cell comprises a nucleic acid sequence encoding an early protein of adenovirus or a functional part, derivative or analogue thereof, wherein said early protein of adenovirus is E1 or E2A.

37. A plant or a non-human animal comprising a plant or non-human animal having a cell of claim 33.

38. The plant or a non-human animal according to claim 37, wherein said plant or non-human animal is transgenic.

39. A cell comprising a cell recognized by a proteinaceous molecule of claim 1, wherein said cell exposes at least one matricryptic epitope on fibronectin.

40. The method according to claim 26, wherein angiogenesis is detected or inhibited.

41. The method according to claim 26, further comprising detecting onset of angiogenesis or a physical condition associated with angiogenesis, wherein said individual is a human subject.

42. The method according to claim 26, further comprising treating a disease in said individual with a therapeutically effective amount of said polypeptide.


SEQUENCE LISTING (9 sequences)

SEQ ID NO: 1 - protein, 22 aa, Artificial
  CDR3 region derived from a heavy chain
EDTAVYYCARNPFQSSFDYWGQ

SEQ ID NO: 2 - protein, 27 aa, Artificial
  CDR3 region derived from a light chain
EDFATYYCSQFSTMPGGFGQGTKVEIK

SEQ ID NO: 3 - protein, 8 aa, Artificial
  Antigen recognized by FibMab
AFTDVDVD

SEQ ID NO: 4 - protein, 11 aa, Artificial
  Antigen recognized by FibMab
IPGHLNSYTIK

SEQ ID NO: 5 - protein, 10 aa, Artificial
  Antigen recognized by FibMab
SSPVTGYRVT

SEQ ID NO: 6 - DNA, 44 bp, Artificial
  HAVT+HindIII primer
aagcttcccataaggaattcggatcccacgatggcatgccctgg

SEQ ID NO: 7 - DNA, 17 bp, Artificial
  M13 primer
gtaaaacgacggccagt

SEQ ID NO: 8 - DNA, 30 bp, Artificial
  Vk1a
tgtgacatcgagctcacccagtctccatcc

SEQ ID NO: 9 - DNA, 53 bp, Artificial
  Jkpan primer
ttctcgacttgcggccgcaaagtgcacttacgtttgatctccaccttggtccc